(19) United States
(12) Patent Application Publication
(10) Pub. No.: US 2008/0206118 A1
(43) Pub. Date: Aug. 28, 2008

(54) Steriliser

(75) Inventor: Peter Smith, Leeds (GB)

Correspondence Address: CHOATE, HALL & STEWART LLP, TWO INTERNATIONAL PLACE, BOSTON, MA, 02110, US

(73) Assignee: jackel International Limited, Cramlington (GB)

(21) Appl. No.: 11/883,313

(22) PCT Filed: Jan. 27, 2006
(86) PCT No.: PCT/GB2006/000273

§ 371 Date: May 12, 2008

(30) Foreign Application Priority Data
Jan. 31, 2005 (GB) 0501946.8

(51) Int. Cl. A61L 2/00

(52) U.S. Cl. 422/292

(57) ABSTRACT

A sterilizer comprising a sterilization chamber (12) and a cover (13). The cover is moveable between an open position and a closed position. When in the closed position, items may be sterilized within the chamber. When moved in the open position, the cover provides a sterile work surface on which to place the sterilized items.

The present invention relates to a sterilizer and in particular a steam sterilizer for sterilizing baby feeding bottles, teats, soothers and the like.

Various known sterilizing units exist all of which rely upon heating a reservoir of water in a covered chamber to produce the steam required for the sterilization process. The water can be heated using an integral electrical heating element, or alternatively units exist that allow the water to be heated by placing the entire sterilizing unit in a microwave oven. The items to be sterilized can be placed directly in the reservoir, or in a separate chamber which allows the ingress of steam. When the items have been subjected to steam for the required period of time, the user opens the unit and removes the sterilized items.

Various problems exist with known arrangements. Following sterilization of the items, the user must remove the items by opening the lid of the unit. When the user has removed the sterilized items from the unit they must place them on, for example, a work surface so as to prepare the items prior to feeding. If the work surface has not previously been sterilized then the items may become contaminated therefore requiring re-sterilization.

The invention is set out in the claims. The sterilizing unit has a lid forming a work surface which will be inherently sterile after operation. Preferably, the lid has a double hinge, having two axes of rotation, allowing the lid to open so that it can be laid flat adjacent to the unit thereby providing the sterile work surface.

Embodiments of the invention will now be described by way of example, with reference to the following drawings, of which:

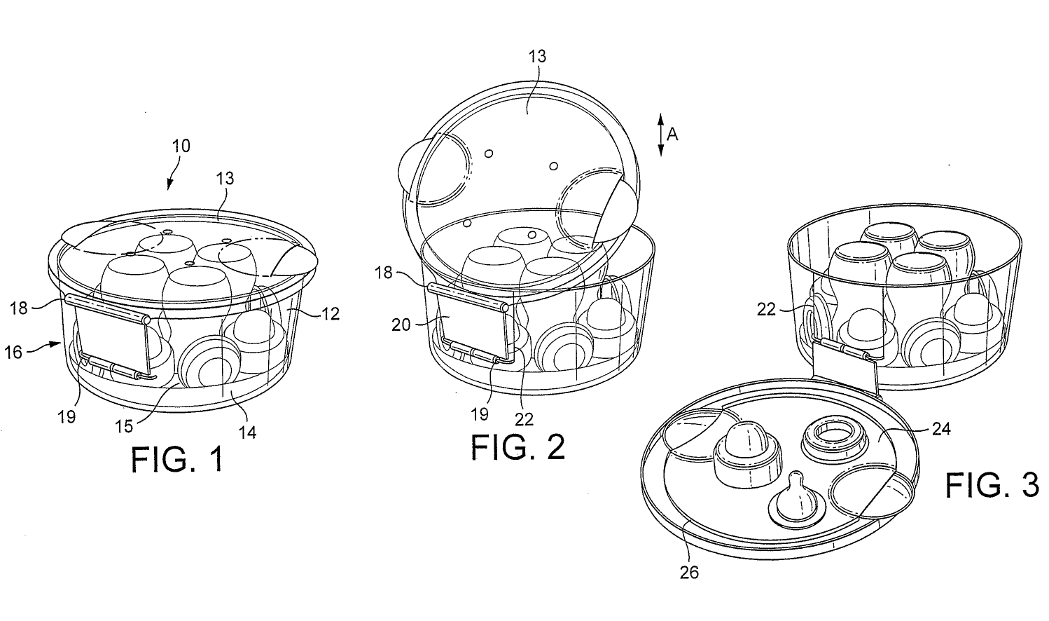

FIG. 1 shows a first perspective view of the sterilization unit embodying the present invention;

FIG. 2 shows a perspective view of the sterilization unit with the lid partially open, and

FIG. 3 shows a perspective view of the sterilization unit with the lid completely open and functioning as a work top.

Referring now to FIG. 1, the sterilizing unit 10 according to the present invention includes a sterilizing chamber 12 having a water reservoir 14. A perforated dividing floor 15, on which the articles to be sterilized are placed, divides the water reservoir 14 from the sterilizing chamber 12. The sterilizing unit 10 further includes a lid 13 which seals the sterilizing chamber 12 when in use. The lid 13 is attached to the chamber 12 using a double hinge mechanism 16. The lid 13 portion may additionally include a clip type handle or handles to assist with opening the lid 13, which may also lock the lid 13 to prevent unwanted opening of the sterilizing unit when in use. To relieve the build up of steam pressure in the chamber during use, the lid 13 may also include a safety valve (not shown) which opens at a predetermined pressure. Additionally, a rim 26 extends along the circumference of the inside face 24 of the lid.

The double hinge mechanism 16 comprises two hinges 18, 19, each hinge defining an axis of rotation. The first hinge 18, defining a first axis of rotation, allows the lid portion 13 to be placed and removed on the sterilizing chamber 12, as indicated by the direction of arrow A in FIG. 2. That is, the lid portion 13 is free to move through at least 180 degrees about said first axis of rotation of the first hinge 18. The first hinge 18 is fixed on one side to the lid portion 13 at its extreme edge and a hinge connecting member 20 on its other side. The hinge connecting member 20 is further connected to a second hinge 19, which is in turn fixed to the outside of the sterilization chamber 12, approximately half way down its length. The second hinge 19, is parallel to the first hinge 18. The second hinge 19 is free to rotate about 180 degrees such that when this lid 13 is removed from the sterilizing chamber 12, the lid 13 can be further rotated about the second hinge 19 so that it can be laid flat adjacent to the sterilizing chamber 12, as shown in FIG. 3. The first hinge 18 and connecting member 20 can further act as a locking mechanism by connecting with the a protruding rim 22 located on the side of sterilization chamber.

In operation, the user fills the reservoir 14 with water and heats the sterilization unit in the microwave so as to produce the steam required to complete the sterilization process. When sterilization is complete, usually several minutes after the water in the reservoir 14 has begun to boil, the sterilization unit 10 is removed from the microwave using the handles. Referring to FIG. 2, the lid 13 of the sterilization unit 10 is then removed from the opening of the sterilization chamber 12 by unclipping the handles upwards and rotating the lid 13, about the first hinge 18. In this way the lid 13 and handles act to shield the user from the escaping residual steam that has built up in the sterilization chamber 12 during the sterilization process. Moreover, by opening the lid 13 in this manner any condensed steam that has built up on the inside surface of the lid 13 will safely run away from the user along the rim into the sterilization chamber 12.

As shown in FIG. 3, when the steam has safely escaped and the user wishes to remove the sterilized articles from the sterilization chamber 12, the lid 13 is then lowered to the work surface by rotating the connecting member 20 through 180 degrees, about the second hinge 19, such that the lid 13 lies inside face 24 up adjacent to the sterilization chamber 12. In this way, the internal surface of the lid 13, which has been subject to steam sterilization, provides a sterile work surface on to which the sterilized articles can be placed.

In an alternative embodiment which does not require microwave heating of the water, the sterilization unit 10 can include an electric heating element (not shown), similar to that used in a domestic kettle, placed in the reservoir 14. In use, the reservoir 14 is filled with water covering the heating element. The heating element, when connected to the electrical supply, heats the water which in turn provides the steam required to carry out sterilization. An optional switch can be provided to turn the heating element on or off as required. An additional timer, or temperature sensor can be employed for switching the unit off automatically following sterilization.

It will be appreciated that the sterilizing unit can be formed of any appropriate material such as for example polypropylene PP or polycarbonate PC, and can be any appropriate shape, which allows placement of the bottles, teats etc. in the chamber. The sterilizing unit can be formed by any appropriate process, such as injection moulding, compression moulding or two-shot moulding. The lid and hinges and chamber can be produced and assembled separately, or alternatively the can be moulded as a complete unit, providing thinned portions as “living” hinges to allow the necessary degree of rotation required to open, close and lower the lid.

1. A sterilizer comprising a sterilization chamber having an opening and a cover mounted between an open position and a closed position, in which the cover forms a work surface in an open position.

2. A sterilizer as claimed in claim 1 further comprising a double hinge portion openably mounting said cover on said opening, wherein said double hinge includes a first hinge and a second hinge and a connecting member extending therebetween, said first hinge connects said cover and said connecting member so that said cover can open and close on said opening, and said second hinge connects the connecting member and said sterilization chamber.

3. A sterilizer as claimed in claim 2, in which said second hinge allows said cover to be lowered so that it lays substantially flat, adjacent the sterilization chamber, to form said worksurface.

4. The sterilizer as claimed in claim 2, wherein said first and second hinges allow rotation by 180 degrees.

5. The sterilizer as claimed in claim 2, wherein said first and second hinges are parallel.

6. The sterilizer as claimed in claim 2, wherein said connecting member is substantially rigid.

7. The sterilizer as claimed in claim 1, wherein said cover is a lid.

8. The sterilizer as claimed in claim 1, wherein the cover includes a condensation collecting formation.

9. (canceled)

10. The sterilizer as claimed in claim 2, wherein said cover is a lid.

11. The sterilizer as claimed in claim 3, wherein said cover is a lid.

12. The sterilizer as claimed in claim 4, wherein said cover is a lid.

13. The sterilizer as claimed in claim 5, wherein said cover is a lid.

14. The sterilizer as claimed in claim 6, wherein said cover is a lid.

15. The sterilizer as claimed in claim 2, wherein the cover includes a condensation collecting formation.

16. The sterilizer as claimed in claim 3, wherein the cover includes a condensation collecting formation.

17. The sterilizer as claimed in claim 4, wherein the cover includes a condensation collecting formation.

18. The sterilizer as claimed in claim 5, wherein the cover includes a condensation collecting formation.

19. The sterilizer as claimed in claim 6, wherein the cover includes a condensation collecting formation.

20. The sterilizer as claimed in claim 7, wherein the lid includes a condensation collecting formation.
